(19) United States
(12) Patent Application Publication
(10) Pub. No.: US 2017/0199306 A1
(43) Pub. Date: Jul. 13, 2017

(54) OPTICALLY CLEAR BIOCOMPATIBLE AND DURABLE HYDROPHILIC COATING PROCESS FOR CONTACT LENSES

(71) Applicant: Xiaoxi Kevin Chen, Natick, MA (US)

(72) Inventor: Xiaoxi Kevin Chen, Natick, MA (US)

(21) Appl. No.: 15/405,309

(22) Filed: Jan. 13, 2017

Related U.S. Application Data
(60) Provisional application No. 62/278,035, filed on Jan. 13, 2016.

(51) Int. Cl. G02B 1/04; B05D 5/06; B05D 7/26; A61B 5/00; G02C 11/00; A61B 5/145; A61B 5/1486; B05D 3/04; B05D 3/14

(52) U.S. Cl. CPC G02B 1/043; B05D 3/0413; B05D 3/0493; B05D 5/06; B05D 7/26; B05D 3/147; G02C 11/10; A61B 5/14532; A61B 5/1486; A61B 5/6821; A61B 2562/12

(57) ABSTRACT

The present invention discloses methods for producing an optically clear, biocompatible and durable hydrophilic coating for contact lenses comprising the steps of first applying a polymer coating on the contact lenses by plasma polymerization of monomers containing ethylene glycol groups, followed by incubating the coated contact lenses at an elevated temperature to remove the volatile residual monomers trapped inside the coating. Advantageously, such methods produce an optically clear, biocompatible and durable hydrophilic surface for contact lenses in a dry, solvent-free process.

### CROSS-REFERENCE TO RELATED APPLICATION

This application claims priority of U.S. Provisional Patent Application No. 62/278,035, filed Jan. 13, 2016, the entire contents of which are incorporated by reference herein.

### FIELD OF THE INVENTION

The present invention discloses methods for producing an optically clear, biocompatible and durable hydrophilic coating for contact lenses comprising the steps of first applying a polymer coating on the contact lenses by plasma polymerization of monomers containing ethylene glycol groups, followed by incubating the coated contact lenses at an elevated temperature to remove the volatile residual monomers trapped inside the coating. Advantageously, such methods produce an optically clear, biocompatible and durable hydrophilic surface for contact lenses in a dry, solvent-free process.

### BACKGROUND OF THE INVENTION

Contact lenses are often made of hydrophobic materials, such as silicone for improving oxygen permeability. It is desirable to modify the surface to make it more hydrophilic and lubricious to avoid tear breaking due to the hydrophobic surface and discomfort due to high friction.

Prior arts of imparting hydrophilic property to silicone contact lenses include oxygen plasma treatment. Although the method can render the silicone surface hydrophilic, the surface will undergo hydrophobic recovery with time (Kim et al. “The Mechanisms of Hydrophobic Recovery of Polydimethylsiloxane Elastomers Exposed to Partial Electrical Discharges”, Journal of Colloid and Interface Science 244, 200-207 (2001)). This is thought to be due to the migration of low molecular weight species from the bulk to the surface of the silicone elastomer.

Other prior arts methods of rendering the silicone contact lenses hydrophilic involve solution coating processes where the silicone contact lenses are immersed in a hydrophilic polymer solution to allow for the coating of the hydrophilic polymer. For example, in U.S. Pat. No. 8,944,592, a method is disclosed where the silicone contact lenses are heated in an aqueous solution in the presence of the hydrophilic polymeric material to and at a temperature from about 40° C. to about 140° C. There are a few disadvantage of these prior art methods that involves a solution coating step. One disadvantage is that the silicone substrate needs to be pre-activated either by oxygen plasma, UV/ozone, corona discharges, plasma polymerization, or other methods, which increases the complexity of the surface coating process. For example, in U.S. Pat. No. 8,944,592, the disclosed method requires that the silicone contact lens contains pre-incorporated amino or carboxyl groups before performing the solution coating step. Another disadvantage is that these methods cannot be used for contact lenses that contain components (such as biosensor components) that can be damaged by water or other solvents due to the requirement of the solution coating step.

Plasma polymerization has the ability to produce a polymer coating on the substrates in a dry state. However, prior art plasma polymerization methods have not been able to provide an optically clear and durable hydrophilic coating for contact lenses. Therefore in prior art methods plasma polymerization is often used as an intermediate step before a solution coating step, which results in a complicated coating process and incompatibility with substrates that contain water-sensitive components. Therefore it is desirable to provide a plasma polymerization method that is able to produce a durable hydrophilic coating without the use of solution coating steps.

### SUMMARY OF THE INVENTION

A method is disclosed herein for applying an optically clear, biocompatible, and durable hydrophilic coating on contact lenses using plasma polymerization of monomer compounds containing ethylene glycol groups, followed by incubation of the coated contact lenses at an elevated temperature to remove residual monomer compounds trapped in the polymer coating during the plasma polymerization coating step.

In the first step, the contact lenses are placed in a plasma polymerization reaction chamber. A plasma excitation power is used to generate plasma glow discharge. The plasma glow discharge, in the presence of the vapor of monomer compounds containing ethylene glycol groups, is used to create a polymer layer with cross-linked poly(ethylene glycol) (PEG). This step provide a surface bound PEG layer containing residual monomer compounds.

In the second step, the plasma polymer coated contact lenses are incubated at an elevated temperature, preferably in a vacuum oven or a convection oven, for a period of time. This step allows the residual monomers trapped inside the polymer coating to evaporate and escape the polymer layer. By removing the trapped monomers, the optical clarity of the contact lenses are greatly improved.

One advantage of the disclosed method is that a high quality PEG polymer layer, free of residual monomers, is covalently attached on the surface of the contact lenses, providing durable hydrophilicity and biocompatibility. By using an additional oven incubation step, the optical clarity of the coated contact lenses is significantly improved due to the removal of residual monomers trapped inside the polymer layer.

A further advantage of the disclosed method is that the biocompatible, durable hydrophilic and optically clear coating is formed in a dry state without the use of any liquid solution or solvent. This is advantageous for coating devices with electronic and/or biosensor components.

These and other features of the invention will be better understood through a study of the following detailed description and accompanying drawings.

### BRIEF DESCRIPTION OF THE FIGURES

FIG. 1 is a drawing representing the subject invention coating process for contact lenses. In the first step, the lens is coated by plasma polymerization of compounds containing ethylene glycol groups. In the second step, the lens is incubated at an elevated temperature to remove the residual monomers trapped in the coating.

FIG. 2 is a chart comparing the clarity of the contact lenses soaked in water for up to 24 hours. The contacted lenses are either coated with plasma polymerized PEG without post coating oven treatment, or coated with plasma polymerized PEG with post coating oven treatment to remove residual monomers.

FIG. 3 is a chart comparing the hydrophilicity of the contact lens surface. The hydrophilicity of the surface is characterized by water contact angle measurement using a 5 microliter water droplet. The contacted lenses are either uncoated, coated with plasma polymerized PEG without post coating oven treatment, or coated with plasma polymerized PEG with post coating oven treatment to remove residual monomers.

### DETAILED DESCRIPTION OF THE INVENTION

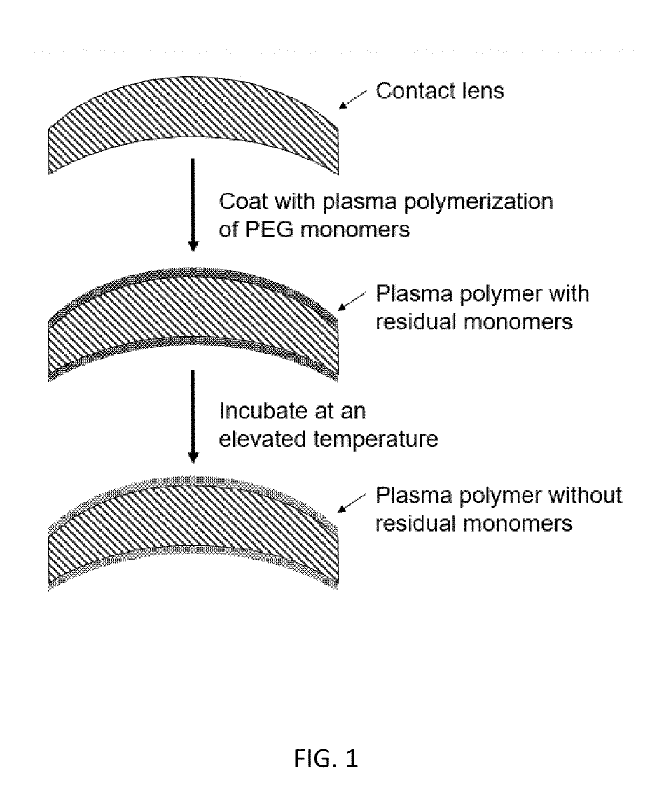

With reference to FIG. 1, a contact lens is depicted of comprising a top surface and bottom surface. Both surfaces are coated by plasma polymerization of monomer compounds containing ethylene glycol groups to form a covalently immobilized layer of PEG polymer containing residual monomers. In the second step, the contact lens is incubated at an elevated temperature to remove residual monomers trapped in the polymer layer.

Any known technique can be used to generate plasma. The plasma may be generated using AC or DC power, radio-frequency (RF) power or micro-wave frequency power. Preferably, the plasma system uses a single radio-frequency (RF) power supply; typically at 13.56 MHz. The plasma system can either be capacitively coupled plasma, or inductively coupled plasma.

Many compounds containing ethylene glycol groups can be used for plasma polymerization. Preferably, the compounds are non-reactive (except when activated by plasma ionization) and non-toxic. Examples of such compounds include Tri(ethylene glycol) monoethyl ether (CH₃CH₂(OCH₂CH₂)₃OH) or Tri(ethylene glycol) monomethyl ether (CH₃(OCH₂CH₂)₃OH).

Any known technique can be used to provide an elevated temperature for the evaporation and removal of residual monomers trapped in the polymer layer. Preferably, a vacuum oven or a convection oven is used for this step.

### EXAMPLES

### Example A

Contact lenses made of silicone elastomer are placed in a plasma polymerization reactor and subsequently coated with a 13.56 Hz radiofrequency plasma glow discharge in the presence of the vapor of Tri(ethylene glycol) monoethyl ether (CH₃CH₂(OCH₂CH₂)₃OH). After plasma polymerization coating, the contact lenses are incubated in a vacuum oven set at 70-80° C. for more than 8 hours.

### Example B

Contact lenses made of silicone elastomer are placed in a plasma polymerization reactor and subsequently coated with a 13.56 Hz radiofrequency plasma glow discharge in the presence of the vapor of Tri(ethylene glycol) monoethyl ether (CH₃CH₂(OCH₂CH₂)₃OH). After plasma polymerization coating, the contact lenses are incubated in a convection oven set at 70-80° C. for more than 8 hours.

Example C

Contact lenses made of silicone elastomer are placed in a plasma polymerization reactor and subsequently coated with a 13.56 Hz radiofrequency plasma glow discharge in the presence of the vapor of Tri(ethylene glycol) monomethyl ether (CH₃(OCH₂CH₂)₃OH). After plasma polymerization coating, the contact lenses are incubated in a vacuum oven set at 80° C. for more than 8 hours.

### Example D

Contact lenses made of silicone elastomer are placed in a plasma polymerization reactor and subsequently coated with a 13.56 Hz radiofrequency plasma glow discharge in the presence of the vapor of Tri(ethylene glycol) monomethyl ether (CH₃(OCH₂CH₂)₃OH). After plasma polymerization coating, the contact lenses are incubated in a convection oven set at 70-80° C. for more than 8 hours.

### Example E

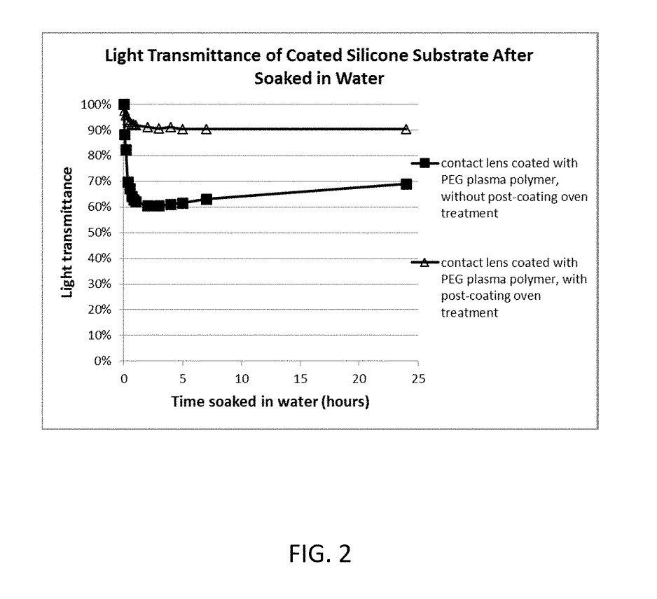

Silicone contact lenses coated with plasma polymerized PEG polymer with or without post-coating treatment to remove residual monomers are compared for optical clarity when soaked in water. Each lens is placed in a quartz cuvette filled with water, and the light transmittance through the lens at 550 nm is monitored using a UV-Vis spectrometer for up to 24 hours. As can be seen in FIG. 2, the light transmittance of the coated lens without post-coating treatment decreased significantly to approximately 60% after soaking in water for a few hours. In contrast, the light transmittance of the coated lens with post-coating treatment remains at >90% throughout 24 hours of soaking.

### Example F

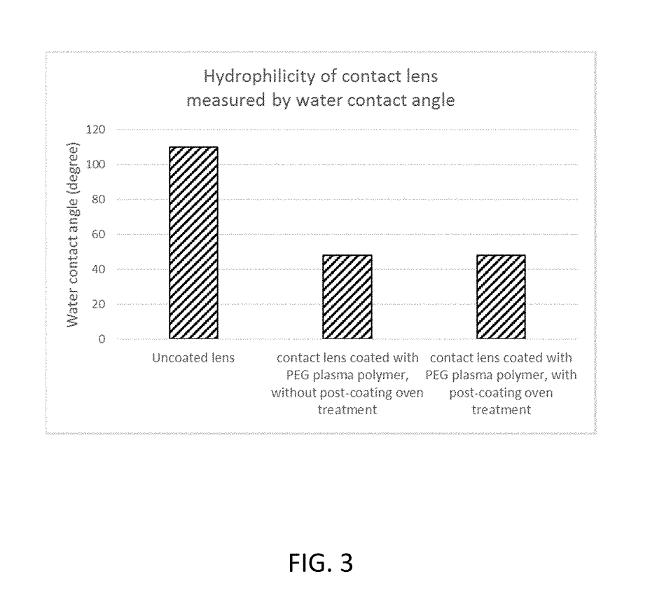

Silicone contact lenses coated with plasma polymerized PEG polymer with or without post-coating treatment to remove residual monomers are compared to uncoated lenses for hydrophilicity. The hydrophilicity of the surface is characterized by water contact angle measurement using a 5 microliter water droplet, and the contact angle measurement is performed after soaking the lenses in water for 1 hour. As can be seen in FIG. 3, the plasma polymerized PEG coating significantly improves the hydrophilicity (decreases the contact angle) compared to the uncoated lenses. Furthermore, the post-coating treatment does not affect the hydrophilicity of the coated contact lenses.

As will be appreciated by those skilled in the art, the subject invention can be used to produce a durable hydrophilic coating. By way of non-limiting example, the subject invention can be used to prepare surfaces of contact lenses made of silicone material, including contact lenses that contain electronic components and/or biosensing components such as glucose sensing enzymes.

What is claimed is:

1. A method for producing a durable hydrophilic and optically clear coating for contact lens comprising the sequential steps of
(i) applying a polymer coating on said contact lens using plasma polymerization of monomer compounds, wherein at least one monomer compound contains ethylene glycol group;
(ii) incubating said contact lens at an elevated temperature to remove excess volatile monomers trapped in the polymer layer.

2. A method of claim 1, wherein said step (ii) is performed in a vacuum oven set at a temperature of higher than 30° C.

3. A method of claim 1, wherein said step (ii) is performed in a convection oven set at a temperature of higher than 30° C.

4. A method of claim 1, wherein said contact lens contains electronic components.

5. A method of claim 1, wherein said contact lens contains biosensor components.

6. A method of claim 1, wherein said contact lens contains biosensor enzymes.

7. A method of claim 1, wherein said contact lens contains glucose sensor components.
